(19) United States
(12) Patent Application Publication
(10) Pub. No.: US 2019/0029895 A1
(43) Pub. Date: Jan. 31, 2019

(54) ELASTOMERIC FILM COMPOSITE AND MANUFACTURING METHOD THEREOF

(71) Applicant: Web-Pro Corporation, Kaohsiung City (TW)

(72) Inventors: CHENG-CHUNG CHIU, Kaohsiung City (TW); PING-SEN LIAO, Kaohsiung City (TW)

(21) Appl. No.: 15/658,426

(22) Filed: Jul. 25, 2017

(51) Int. Cl. A61F 13/49; B32B 25/10; B32B 5/02; B29C 47/06; B29C 47/00; B29C 55/14

(52) U.S. Cl. CPC A61F 13/49009; B32B 25/10; B32B 5/022; B29C 47/065; B29K 2023/12; B29C 55/143; B29C 47/004; B32B 2555/02; B29C 47/0057

(57) ABSTRACT

The present invention relates to an elastomeric film composite, which comprises: at least one upper surface layer, at least one intermediate layer, and at least one lower surface layer. Material surface compatibility is selectively made poor and is used to pull open a surface gap between layers through applying an extension process without application of hot pressing so that the elastomeric film composite is made to have an embossed surface structure in the form of cloth.

### (a) TECHNICAL FIELD OF THE INVENTION

The present invention relates generally to a personal sanitary article or a covering material, and more particularly to an elastomeric film composite applicable to a disposable sanitary article featuring expansion between surfaces of layers by means of compatibility difference between material through an extension process exhibiting a structure free of hot pressing spots that enhances hand feeling of material softness and an interlayer structure for easy recyclability that helps reduce scrape.

### (b) DESCRIPTION OF THE PRIOR ART

An elastomeric film composite is a material that is made of raw materials of rubbers and/or plastics through an injection or extrusion process to form elastic materials of different outside configurations for applications in different fields. The elastomeric film shows a property of elasticity capable of springing back and may readily undergo variation through elasticity thereof to suit different types of movement of human bodies, showing property of human body conformableness and reliability of being fitted to and fixed to human body. Thus, it is an important issue for application to elastic composites for personal sanitary materials.

Prior studies of elastic non-woven fabric that are known, such as Taiwan Patent No. 333569, which discloses a production method of longitudinal and transverse elastic non-woven fabric through thermal and mechanical processing, in which a thermoplastic and a part of mixed non-thermoplastic non-woven is processed for thermally adhering, and extension is made with a low extension rate to improve softness and touch conformableness of cloth and also achieve elasticity having high commercial value to make fabric that is fitted to applications where softness and extendible non-woven fabric. After processing, unidirectional elasticity is generated, two designs being available for achieving mechanical longitudinal or fabric widthwise elasticity. Virtually all kinds of non-woven fabric can be processed in this way to provide desired softness and extendible elasticity, provided it contains more than 70% thermoplastic fibers. Further, Taiwan Patent No. I271455 discloses an elastic composite fabric, which comprises at least one non-woven fabric, at least another fabric, and a thread band of elastic threads. The thread band is arranged between the non-woven fabrics and said another fabric. The non-woven fabric is thermally fused and jointed to another fabric showing a predetermined pattern, and the elastic threads are buried, in a tensioned condition, in a fusion joint spot between the non-woven fabric and said another fabric at a selected site. Such a composite can be used to make sanitary articles, particularly diapers, including diaper pants.

Prior studies on toothed roller snapping engagement to form surface projections are known, such as Taiwan Patent No. I282383, which discloses a sheathed composite fiber that is formed by combining polyester and a polyolefin core, or a polyester fiber is used, in which a molten high molecule film is formed through lamination and coating on a bottom of a loop fiber net layer, followed by pressing with inter-engaging toothed rollers to form felt non-woven fabric, where the felt has a property of high resistance to compression.

Prior studies concerning production of ultrasonic wave welded elastic non-woven fabric are known, such as Taiwan Patent No. I405660, which discloses a structure that comprises an elastic film having one side adhered, through ultrasonic welding, to a non-fabric article and showing improved puffiness and improved softness and hand touch feeling, and providing an elastic laminate having a relatively large rolling capability, wherein joint spots comprise a flat joint area occupying an area that is not greater than 30% of an entire area. The prior art requires ultrasonic welding or hot pressing applied to a surface of a film or a non-woven fabric to form, through pressing, jointing spots or areas, and then an extension process is applied, with the jointing spots as fixed points, to pull and extend the inelastic material, this being also referred to as an activation process. The jointing spots, due to being pressing, become pressed jointing points. Such locations are in fact suffering various drawbacks, such as inelasticity, low moisture absorbability, and hard hand touch feeling. Thus, further improvements are necessary.

### SUMMARY OF THE INVENTION

Based on the deficiencies of the production of the known elastomeric film composite, the present invention aims to provide an elastomeric film composite and a manufacturing method to alleviate the drawbacks of the prior art. The present invention provides an elastomeric film composite, which comprises: at least one upper surface layer, at least one intermediate layer, and at least one lower surface layer, wherein material surface compatibility difference is used, in combination with a co-extrusion process or a lamination process and an extension process, to pull open a surface gap between layers through applying the extension process without first applying hot pressing so that the elastomeric film composite is made to have an embossed surface structure in the form of cloth.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a view showing an un-extended elastic intermediate layer microporous or non-porous film composite structure according to the present invention.

FIG. 2 is a view showing an extended elastic intermediate layer microporous or non-porous film composite microporous or non-porous film composite structure according to the present invention.

FIG. 3 is a view showing an un-extended elastic lower surface layer microporous or non-porous film composite structure according to the present invention.

FIG. 4 is a view showing an extended elastic lower surface layer microporous or non-porous film composite structure according to the present invention.

FIG. 5 is a view showing a destructively extended elastic intermediate layer microporous or non-porous film composite microporous or non-porous film composite structure according to the present invention.

FIG. 6 is a view showing a destructively extended elastic lower surface layer microporous or non-porous film composite structure according to the present invention.

FIG. 7 is a view showing an un-extended non-woven fabric elastic intermediate layer microporous or non-porous film composite structure according to the present invention.

FIG. 8 is a view showing an extended non-woven fabric elastic intermediate layer microporous or non-porous film composite structure according to the present invention.

FIG. 9 is a view showing an un-extended elastic intermediate layer and inelastic non-woven fabric lower surface layer microporous or non-porous film composite structure according to the present invention.

FIG. 10 is a view showing an extended elastic intermediate layer and inelastic non-woven fabric lower surface layer microporous or non-porous film composite structure according to the present invention.

FIG. 11 is a view showing an un-extended elastic intermediate layer and inelastic non-woven fabric upper and lower surface layer microporous or non-porous film composite structure according to the present invention.

FIG. 12 is a view showing an extended elastic intermediate layer and inelastic non-woven fabric upper and lower surface layer microporous or non-porous film composite structure according to the present invention.

FIG. 13 is a view showing an un-extended elastic lower surface layer and inelastic non-woven fabric intermediate layer microporous or non-porous film composite structure according to the present invention.

FIG. 14 is a view showing an extended elastic lower surface layer and inelastic non-woven fabric intermediate layer microporous or non-porous film composite structure according to the present invention.

FIG. 15 is a view showing an un-extended elastic lower surface layer and inelastic non-woven fabric upper surface layer microporous or non-porous film composite structure according to the present invention.

FIG. 16 is a view showing an extended elastic lower surface layer and inelastic non-woven fabric upper surface layer microporous or non-porous film composite structure according to the present invention.

FIG. 17 is a microscopic picture showing a film surface of an elastic intermediate layer microporous film composite having a small-sized surface gap according to the present invention.

FIG. 18 is a microscopic picture showing a film surface of an elastic intermediate layer microporous film composite having a medium-sized surface gap according to the present invention.

FIG. 19 is a microscopic picture showing a film surface of an elastic intermediate layer microporous film composite having a large-sized surface gap according to the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

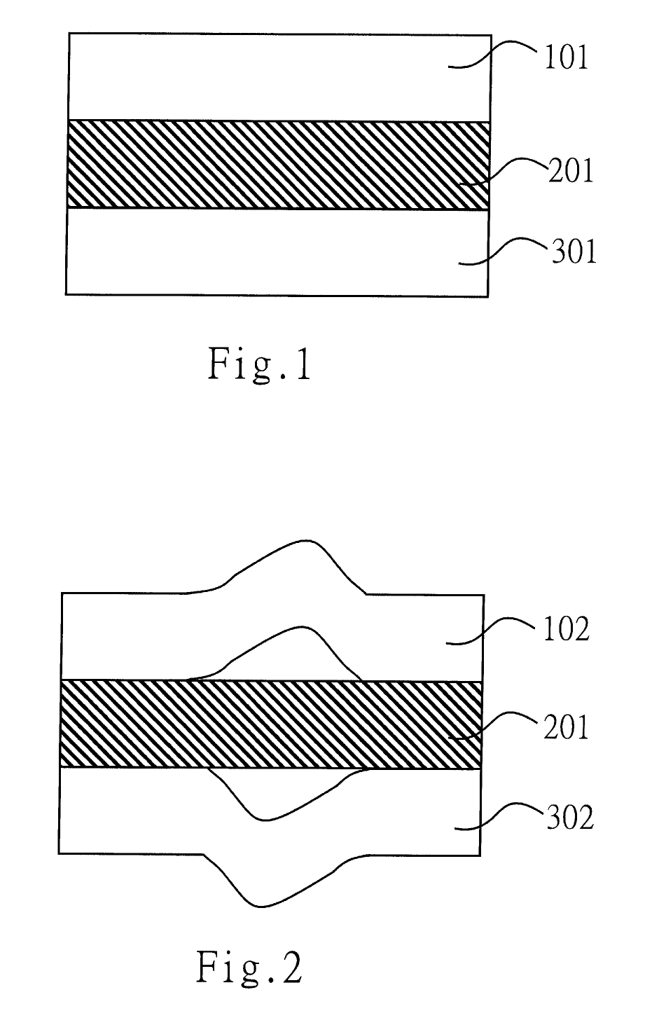

Referring to FIG. 1, a view is provided to show an un-extended elastic intermediate layer microporous or non-porous film composite structure according to the present invention, comprising: an upper surface layer 101, being an un-extended inelastic microporous or non-porous film, which is adjacent to a first surface of an intermediate layer 201; an intermediate layer 201, being an elastic microporous or non-porous film, which comprises a first surface and a second surface, arranged between the upper surface layer 101 and a lower surface layer 301; and a lower surface layer 301, being an un-extended inelastic microporous film. A structure of the un-extended elastic intermediate layer microporous or non-porous film composite, after an extension process, is converted into an extended elastic intermediate layer microporous or non-porous film composite structure according to the present invention as shown in FIG. 2. After the extension, an external tension force is removed and the intermediate layer 201 is made, as an elastic microporous or non-porous film, spring back and exhibit a stabilized state, while the upper surface layer 102, which becomes an extended inelastic microporous or non-porous film and the lower surface layer 302, which becomes an extended inelastic microporous or non-porous film, show no property of springing back and undergo deformation as being extended by the extension force, such that material surface compatibility differences (among the layers) are used to pull open a surface gap between layers through applying an extension process without application of a hot pressing process, wherein the upper surface layer 102 and the intermediate layer 201 are partly separated from each other and the lower surface layer 302 and the intermediate layer 201 are partly separated from each other such that an interlayer surface gap having a size of 0.5-1000 micrometers is formed and the elastomeric film composite is made to have an embossed surface structure in the form of cloth. The intermediate layer 201 is an elastic material of which a type is an elastic non-porous film, an elastic microporous film, or an elastic perforated film, and is selected as one of Hytrel (Polyester Elastomer), TPU (Thermoplastic Polyurethane), SEBS (Styrene Ethylene Butylene Styrene), SIS (Styrene Isoprene Styrene), SBS (Styrene Butadiene Styrene), polypropylene elastomer, polyethylene elastomer, elastic nylon (Polyamide Elastomer), or a mixture of the above materials. The upper surface layer 102 or the lower surface layer 302 is an inelastic material of which a type can be an inelastic non-porous film, an inelastic microporous film, or an inelastic perforated film and is selected as one of PET (Polyester), PP (Polypropylene), PE (polyethylene), PS (Polystyrene), Nylon (Polyamide), or a mixture of the above materials. Further, the upper surface layer 102 or the lower surface layer 302 is preferably of a material that comprises PP or a mixture of PP. Further, the upper surface layer 102, the intermediate layer 201, or the lower surface layer 302 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 201 has a thickness that is 5-70% of the entire thickness; and the lower surface layer 302 has a thickness that is 5-70% of the entire thickness.

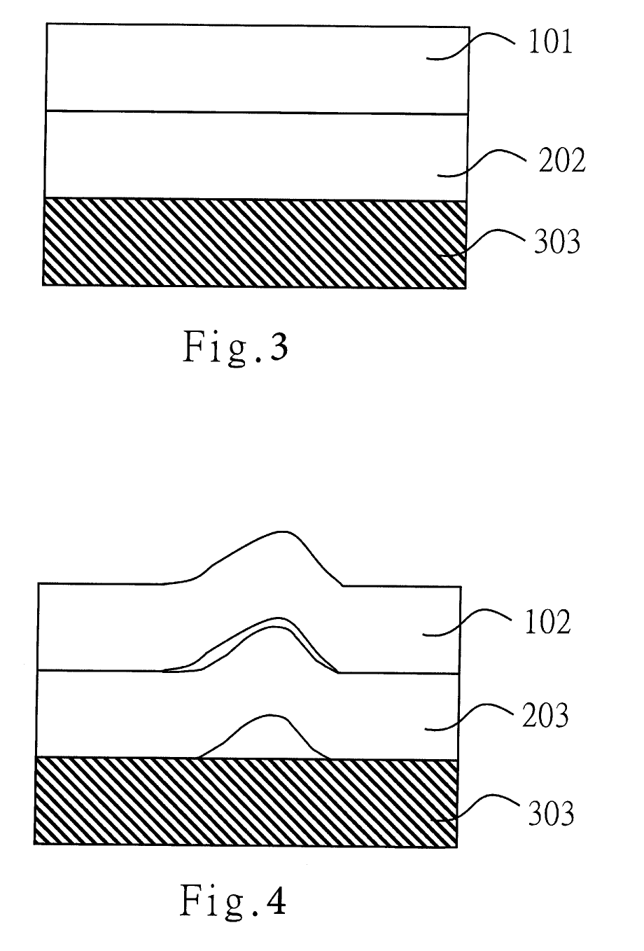

FIG. 3 is a view of an un-extended elastic lower surface layer microporous or non-porous film composite structure according to the present invention, comprising: an upper surface layer 101, being an un-extended inelastic microporous or non-porous film, which is adjacent to a first surface of an intermediate layer 202; an intermediate layer 202, being an un-extended inelastic microporous or non-porous film, which comprises a first surface and a second surface, arranged between the upper surface layer 101 and a lower surface layer 303, and a lower surface layer 303, being an elastic microporous or non-porous film. A structure of the un-extended elastic lower surface layer microporous or non-porous film composite, after an extension process, is converted into an extended elastic lower surface layer microporous or non-porous film composite structure according to the present invention as shown in FIG. 4. After the extension, an external tension force is removed and the lower surface layer 303 is made, as an elastic microporous or non-porous film, spring back and exhibit a stabilized state, while the upper surface layer 102, which becomes an extended inelastic microporous or non-porous film and the intermediate layer 203, which becomes an extended inelastic microporous or non-porous film, show no property of springing back and undergo deformation as being extended by the extension force, such that material surface compatibility differences are used to pull open a surface gap between layers through applying an extension process without application of a hot pressing process, wherein the upper surface layer 102 and the intermediate layer 203 are partly separated from each other and the lower surface layer 303 and the intermediate layer 202 are partly separated from each other such that an interlayer surface gap having a size of 0.5-1000 micrometers is formed and the elastomeric film composite is made to have an embossed surface structure in the form of cloth. The lower surface layer 303 is an elastic material of which a type is an elastic non-porous film, an elastic microporous film, or an elastic perforated film, and is selected as one of Hytrel, TPU, SEBS, SIS, SBS, polypropylene elastomer, polyethylene elastomer, elastic nylon, or a mixture of the above materials. The upper surface layer 102 or the intermediate layer 203 is an inelastic material of which a type can be an inelastic non-porous film, an inelastic microporous film, or an inelastic perforated film and is selected as one of PET, PP, PE, PS, Nylon, or a mixture of the above materials. Further, the upper surface layer 102 or the lower surface layer 303 is preferably of a material that comprises PP, PP elastomer, or a mixture of the above materials. Further, the upper surface layer 102, the intermediate layer 203, or the lower surface layer 303 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 203 has a thickness that is 5-70% of the entire thickness; and the lower surface layer 303 has a thickness that is 5-70% of the entire thickness.

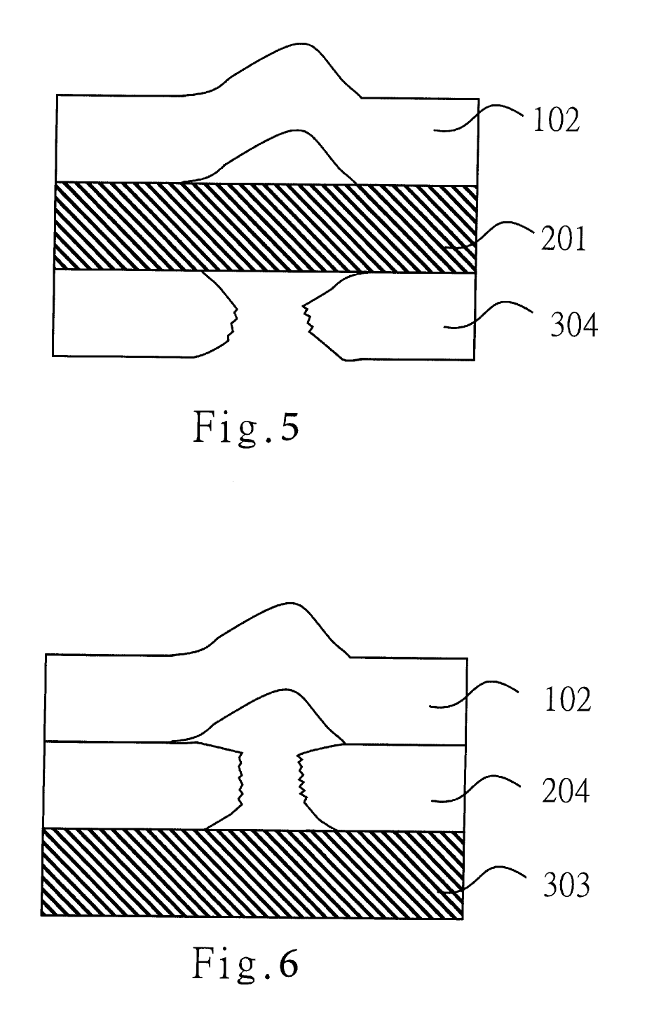

To provide a better understanding of an actual application of the present invention, an example that shows a destructive extension of structure is provided for explanation. FIG. 5 is a view showing a destructively extended elastic intermediate layer microporous or non-porous film composite structure, which after being subjected to an extension process undergoes structure change, and after the extension, an external tension force is removed to make the intermediate layer 201, as an elastic microporous or non-porous film, spring back and exhibit a stabilized state, while an upper surface layer 102, which becomes an extended inelastic microporous or non-porous film, and a lower surface layer 304, which becomes a destructively extended inelastic microporous or non-porous film, show no property of springing back and undergo deformation as being extended by the extension force, wherein the lower surface layer 304 being a destructively extended inelastic microporous or non-porous film is because of inclusion of a material featuring a property of low rate of extension that leads to formation of holes through breaking. Further, FIG. 6 is a view showing a destructively extended elastic lower surface layer microporous or non-porous film composite structure according to the present invention, which is also subjected to an extension process to cause structure change and after the extension, an external tension force is removed to make the lower surface layer 303, as an elastic microporous or non-porous film spring back and exhibit a stabilized state, while an upper surface layer 102, which becomes an extended inelastic microporous or non-porous film, and an intermediate layer 204, which becomes a destructively extended inelastic microporous or non-porous film, show no property of springing back and undergo deformation as being extended by the extension force, wherein the intermediate layer 204 being a destructively extended inelastic microporous or non-porous film is because of inclusion of a material featuring a property of low rate of extension that leads to formation of holes through breaking. Material surface compatibility differences are used to pull open a surface gap between layers through applying an extension process without application of a hot pressing process so that the elastomeric film composite is made to have an embossed surface structure in the form of cloth having large raised bulge. The intermediate layer 201 or the lower surface layer 303 is an elastic material of which a type is an elastic non-porous film, an elastic microporous film, or an elastic perforated film, and is selected as one of Hytrel, TPU, SEBS, SIS, SBS, polypropylene elastomer, polyethylene elastomer, elastic nylon, or a mixture of the above materials. The upper surface layer 102, the lower surface layer 304, or the intermediate layer 204 is an inelastic material of which a type can be an inelastic non-porous film, an inelastic microporous film, or an inelastic perforated film and is selected as one of PET, PP, PE, PS, Nylon, or a mixture of the above materials. Further, the upper surface layer 102, the lower surface layer 304, or the lower surface layer 303 is preferably of a material that comprises PP, PP elastomer, or a mixture of the above materials. Further, the upper surface layer 102, the intermediate layer 201, the intermediate layer 204, the lower surface layer 304, or the lower surface layer 303 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 201 or the intermediate layer 204 has a thickness that is 5-70% of the entire thickness; and the lower surface layer 304 or the lower surface layer 303 has a thickness that is 5-70% of the entire thickness.

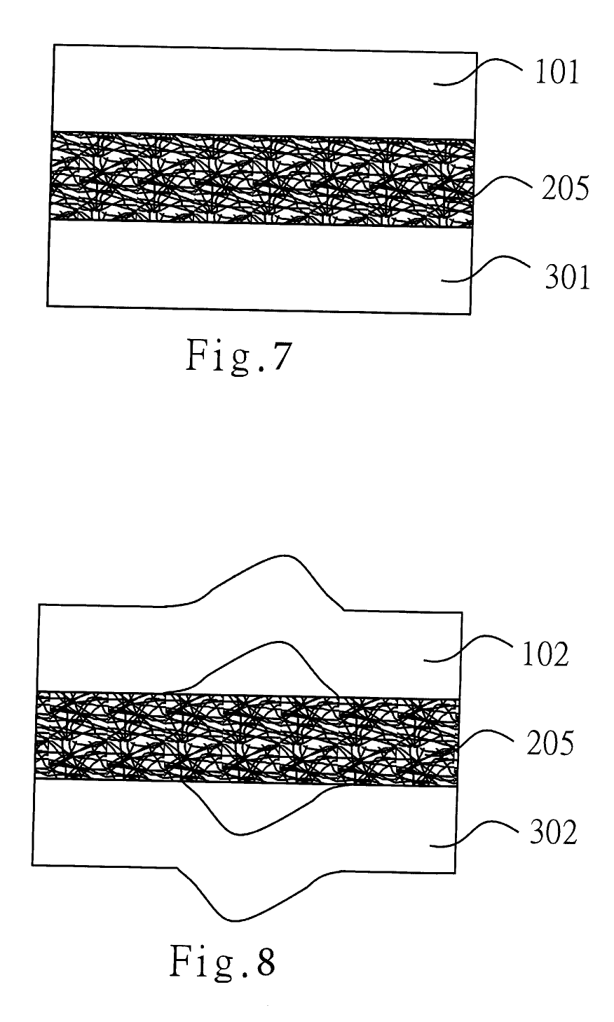

As another embodiment, FIG. 7 is a view showing an un-extended non-woven fabric elastic intermediate layer microporous or non-porous film composite structure, which comprises: an upper surface layer 101, being an un-extended inelastic microporous or non-porous film, which is adjacent to a first surface of an intermediate layer 205; an intermediate layer 205, being non-woven fabric, which comprises a first surface and a second surface, arranged between the upper surface layer 101 and a lower surface layer 301; and a lower surface layer 301, being an un-extended inelastic microporous or non-porous film. A structure of the un-extended non-woven fabric elastic intermediate layer microporous or non-porous film composite, after an extension process, is converted into an extended non-woven fabric elastic intermediate layer microporous or non-porous film composite structure according to the present invention as shown in FIG. 8. After the extension, an external tension force is removed and the intermediate layer 205 is made, as an elastic non-woven fabric, spring back and exhibit a stabilized state, while the upper surface layer 102, which becomes an extended inelastic microporous or non-porous film and the lower surface layer 302, which becomes an extended inelastic microporous or non-porous film, show no property of springing back and undergo deformation as being extended by the extension force, such that material surface compatibility differences are used to pull open a surface gap between layers through applying an extension process without application of a hot pressing process, wherein the upper surface layer 102 and the intermediate layer 205 are partly separated from each other and the lower surface layer 302 and the intermediate layer 205 are partly separated from each other such that an interlayer surface gap having a size of 0.5-1000 micrometers is formed and the elastomeric film composite is made to have an embossed surface structure in the form of cloth. The intermediate layer 302 is an elastic material of which a type is an elastic non-woven fabric and is selected as one of Hytrel, TPU, SEBS, SIS, SBS, polypropylene elastomer, polyethylene elastomer, elastic nylon, or a mixture of the above materials. The upper surface layer 102 or the lower surface layer 302 is an inelastic material of which a type can be an inelastic non-porous film, an inelastic microporous film, or an inelastic perforated film and is selected as one of PET, PP, PE, PS, Nylon, or a mixture of the above materials. Further, the upper surface layer 102 or the lower surface layer 302 is preferably of a material that comprises PP or a mixture of PP. Further, the upper surface layer 102 or the lower surface layer 302 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 302 has a thickness that is 5-70% of the entire thickness; and the lower surface layer 302 has a thickness that is 5-70% of the entire thickness.

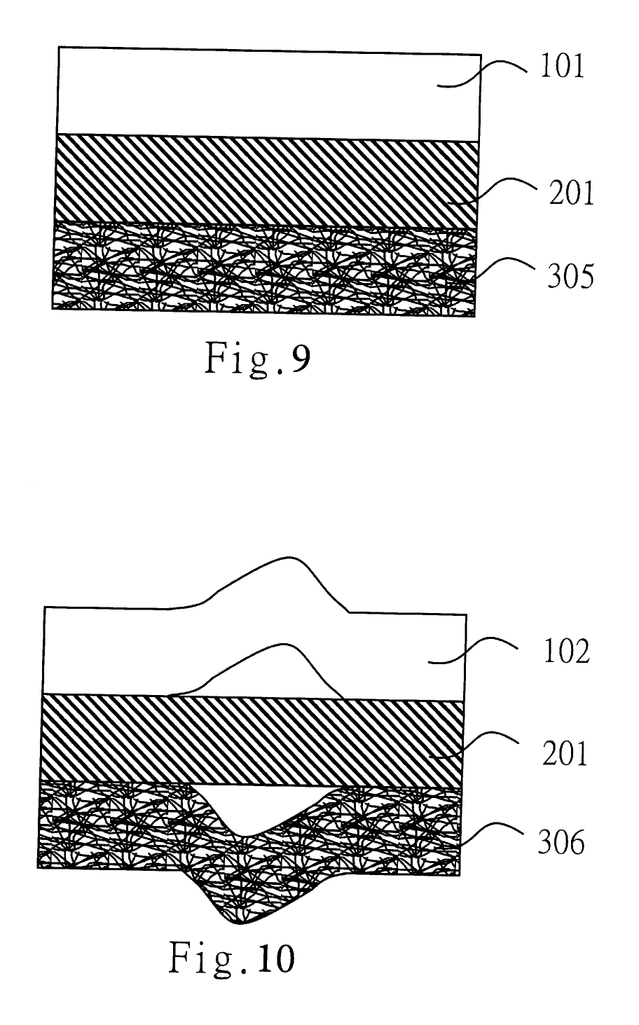

FIG. 9 is a view showing an un-extended elastic intermediate layer and inelastic non-woven fabric lower surface layer microporous or non-porous film composite structure, which comprises: an upper surface layer 101, being an un-extended inelastic microporous or non-porous film in the un-extended condition, which is adjacent to a first surface of an intermediate layer 201; an intermediate layer 201, being an elastic microporous or non-porous film, which comprises a first surface and a second surface, arranged between the upper surface layer 101 and a lower surface layer 305; and a lower surface layer 305, being an un-extended non-woven fabric. A structure of the un-extended elastic intermediate layer and lower surface layer inelastic non-woven fabric microporous or non-porous film composite, after an extension process, is converted into an extended elastic intermediate layer and inelastic non-woven fabric lower surface layer microporous or non-porous film composite structure as shown in FIG. 10. After the extension, an external tension force is removed and the intermediate layer 201 is made, as an elastic microporous or non-porous film, spring back and exhibit a stabilized state, while the upper surface layer 102, which becomes an extended inelastic microporous or non-porous film, and the lower surface layer 306, which becomes an extended inelastic non-woven fabric, show no property of springing back and undergo deformation as being extended by the extension force, such that material surface compatibility differences are used to pull open a surface gap between layers through applying an extension process without application of a hot pressing process, wherein the upper surface layer 102 and the intermediate layer 201 are partly separated from each other and the lower surface layer 306 and the intermediate layer 201 are partly separated from each other such that an interlayer surface gap having a size of 0.5-1000 micrometers is formed and the elastomeric film composite is made to exhibit dual-side effects of having an embossed surface structure in the form of cloth on one side and being a non-woven fabric on an opposite side. The intermediate layer 201 is an elastic material of which a type is an elastic non-woven fabric and is selected as one of Hytrel, TPU, SEBS, SIS, SBS, polypropylene elastomer, polyethylene elastomer, elastic nylon, or a mixture of the above materials. The upper surface layer 102 or the lower surface layer 306 is an inelastic material of which a type can be a non-woven fabric, an inelastic non-porous film, an inelastic macroporous film, or an inelastic perforated film and is selected as one of PET, PP, PE, PS, Nylon, or a mixture of the above materials. Further, the upper surface layer 102 or the lower surface layer 306 is preferably of a material that comprises PP or a mixture of PP. Further, the upper surface layer 102 or the intermediate layer 201 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 201 has a thickness that is 5-70% of the entire thickness; and the lower surface layer 306 has a thickness that is 5-70% of the entire thickness.

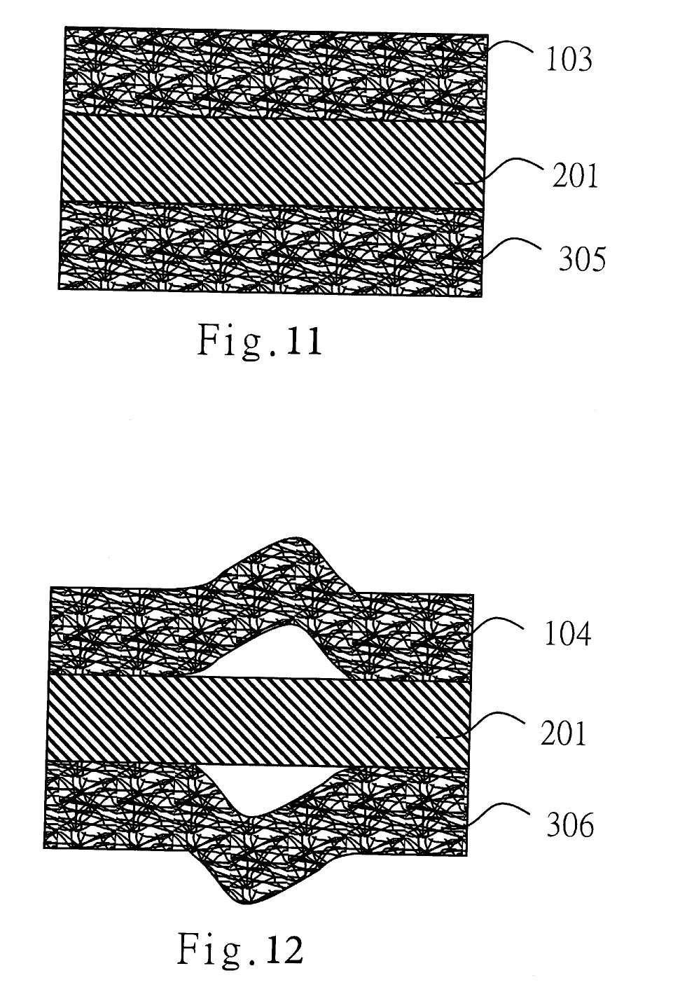

FIG. 11 is a view showing an un-extended elastic intermediate layer and inelastic non-woven fabric upper and lower surface layer microporous or non-porous film composite structure, which comprises: an upper surface layer 103, being an un-extended inelastic non-woven fabric, which is adjacent to a first surface of an intermediate layer 201; an intermediate layer 201, being an elastic microporous or non-porous film, which comprises a first surface and a second surface, arranged between the upper surface layer 103 and a lower surface layer 305; and a lower surface layer 305, being an un-extended inelastic non-woven fabric. A structure of the un-extended elastic intermediate layer and upper and lower surface layer inelastic non-woven fabric microporous or non-porous film composite, after an extension process, is converted into an extended elastic intermediate layer and inelastic non-woven fabric upper and lower surface layer microporous or non-porous film composite structure as shown in FIG. 12. After the extension, an external tension force is removed and the intermediate layer 201 is made, as an elastic microporous or non-porous film spring back and exhibit a stabilized state, while the upper surface layer 104, which becomes an extended inelastic non-woven fabric, and the lower surface layer 306, which becomes an extended inelastic non-woven fabric, show no property of springing back and undergo deformation as being extended by the extension force, such that material surface compatibility differences are used to pull open a surface gap between layers through applying an extension process without application of a hot pressing process, wherein the upper surface layer 104 and the intermediate layer 201 are partly separated from each other and the lower surface layer 306 and the intermediate layer 201 are partly separated from each other such that an interlayer surface gap having a size of 0.5-1000 micrometers is formed and the elastomeric film composite is made to exhibit a surface effect of being puffy non-woven fabric on opposite sides. The intermediate layer 201 is an elastic material of which a type is an elastic non-woven fabric and is selected as one of Hytrel, TPU, SEBS, SIS, SBS, polypropylene elastomer, polyethylene elastomer, elastic nylon, or a mixture of the above materials. The upper surface layer 104 or the lower surface layer 306 is an inelastic material of which a type can be a non-woven fabric and is selected as one of PET, PP, PE, PS, Nylon, or a mixture of the above materials. Further, the upper surface layer 104 or the lower surface layer 306 is preferably of a material that comprises PP or a mixture of PP. Further, the upper surface layer 104 has a thickness that is 5-70% of an entire thickness; the intermediate layer 201 has a thickness that is 5-70% of the entire thickness; and the lower surface layer 306 has a thickness that is 5-70% of the entire thickness.

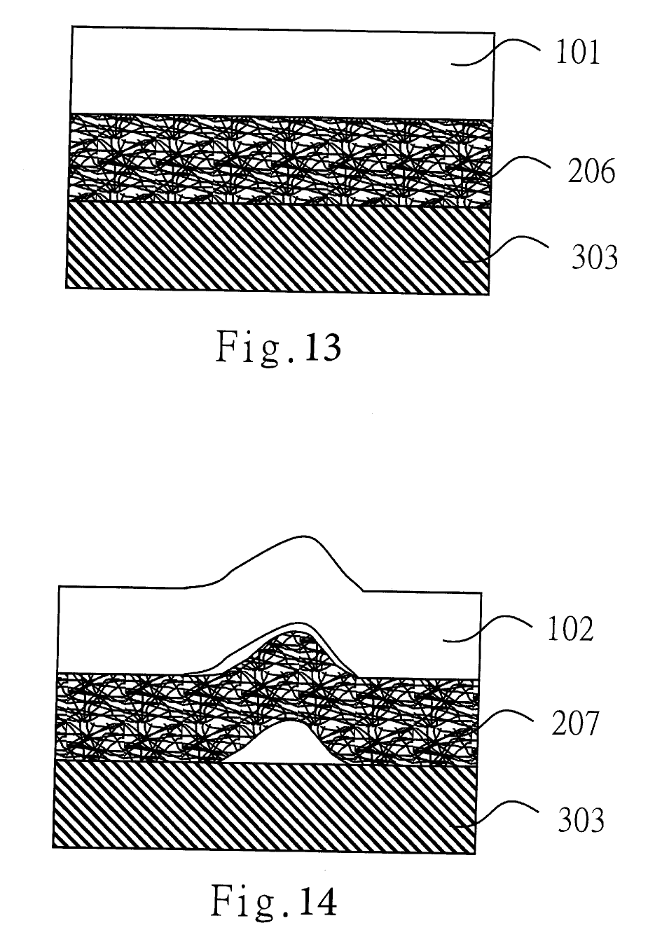

FIG. 13 is a view showing an un-extended elastic lower surface layer and inelastic non-woven fabric intermediate layer microporous or non-porous film composite structure, which comprises: an upper surface layer 101, being an inelastic microporous or non-porous film in the un-extended condition, which is adjacent to a first surface of an intermediate layer 206; an intermediate layer 206, being an un-extended inelastic non-woven fabric, which comprises a first surface and a second surface, arranged between the upper surface layer 101 and the lower surface layer 303; and a lower surface layer 303, being an elastic microporous or non-porous film. A structure of the un-extended elastic lower surface layer and inelastic non-woven fabric intermediate layer microporous or non-porous film composite, after an extension process, is converted into an extended elastic lower surface layer and inelastic non-woven fabric intermediate layer microporous or non-porous film composite structure shown in FIG. 14. After the extension, an external tension force is removed and the lower surface layer 303 is made, as elastic microporous or non-porous film, spring back and exhibit a stabilized state, while the upper surface layer 102, which becomes an extended inelastic microporous or non-porous film, and the intermediate layer 207, which becomes an extended inelastic non-woven fabric, show no property of springing back and undergo deformation as being extended by the extension force, such that material surface compatibility differences are used to pull open a surface gap between layers through applying an extension process without application of a hot pressing process, wherein the upper surface layer 102 and the intermediate layer 207 are partly separated from each other and the lower surface layer 303 and the intermediate layer 207 are partly separated from each other such that an interlayer surface gap having a size of 0.5-1000 micrometers is formed and the elastomeric film composite is made to have an embossed structure in the form of cloth having large raised bulge. The lower surface layer 303 is an elastic material of which a type is an elastic non-woven fabric and is selected as one of Hytrel, TPU, SEBS, SIS, SBS, polypropylene elastomer, polyethylene elastomer, elastic nylon, or a mixture of the above materials. The upper surface layer 102 or the intermediate layer 207 is an inelastic material of which a type can be a non-woven fabric, an inelastic non-porous film, an inelastic microporous film, or an inelastic perforated film and is selected as one of PET, PP, PE, PS, Nylon, or a mixture of the above materials.

Further, the upper surface layer 102 or the lower surface layer 303 is preferably of a material that comprises PP, polypropylene elastomer, or a mixture of the above materials. Further, the upper surface layer 102 or the lower surface layer 303 comprises a material that is added with inorganic powder of calcium carbonate, magnesium carbonate, oxides of aluminum, or oxides of titanium having a weight percentage of 1-75%. The upper surface layer 102 has a thickness that is 5-70% of an entire thickness; the intermediate layer 207 has a thickness that is 5-70% of the entire thickness; and the lower surface layer 303 has a thickness that is 5-70% of the entire thickness.

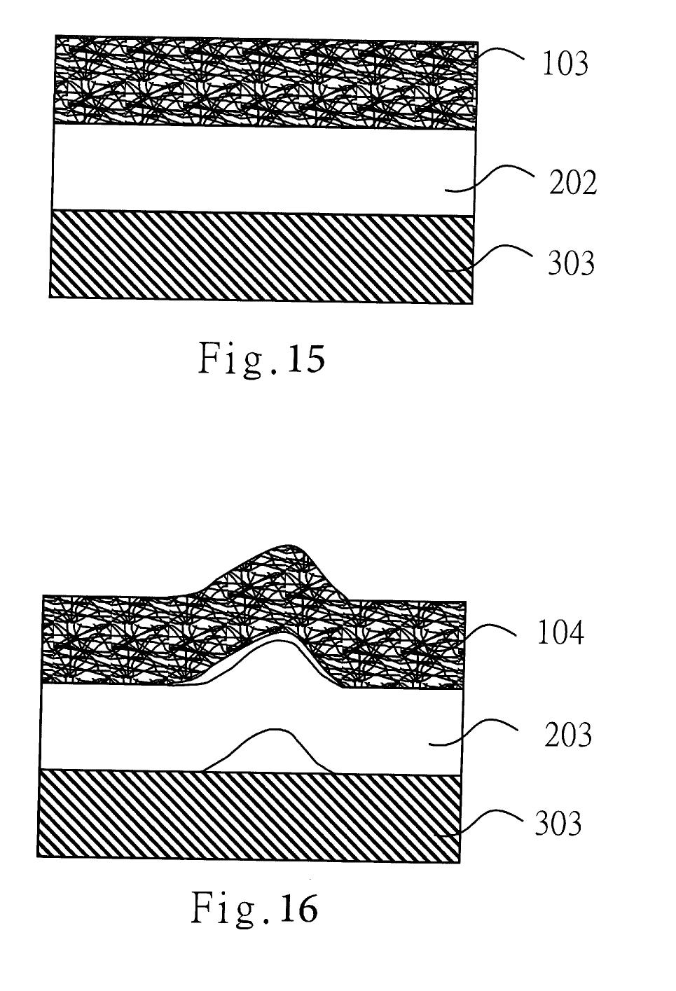

FIG. 15 is a view showing an un-extended elastic lower surface layer and inelastic non-woven fabric upper surface layer microporous or non-porous film composite structure, which comprises: an upper surface layer 101, being an un-extended inelastic non-woven fabric, which is adjacent to a first surface of an intermediate layer 202; an intermediate layer 202, being an un-extended inelastic microporous or non-porous film, which comprises a first surface and a second surface, arranged between the upper surface layer 103 and a lower surface layer 303; and a lower surface layer 303, being an elastic microporous or non-porous film. A structure of the un-extended elastic lower surface layer and inelastic non-woven fabric upper surface layer microporous or non-porous film composite, after an extension process, is converted into an extended elastic lower surface layer and inelastic non-woven fabric upper surface layer microporous or non-porous film composite structure as shown in FIG. 16. After the extension, an external tension force is removed and the lower surface layer 303 is made, as an elastic microporous or non-porous film, spring back and exhibit a stabilized state, while the upper surface layer 104, which becomes an extended inelastic non-woven fabric, and the intermediate layer 203, which becomes an extended inelastic microporous or non-porous film, show no property of springing back and undergo deformation as being extended by the extension force, such that material surface compatibility differences are used to pull open a surface gap between layers through applying an extension process without application of a hot pressing process, wherein the upper surface layer 104 and the intermediate layer 203 are partly separated from each other and the lower surface layer 303 and the intermediate layer 203 are partly separated from each other such that an interlayer surface gap having a size of 0.5-1000 micrometers is formed and the elastomeric film composite is made to exhibit dual-side effects of being fluffy non-woven fabric on one side and being an elastic film in an opposite side. The lower surface layer 303 is an elastic material of which a type is an elastic non-porous film, an elastic microporous film, or an elastic perforated film and is selected as one of Hytrel, TPU, SEBS, SIS, SBS, polypropylene elastomer, polyethylene elastomer, elastic nylon, or a mixture of the above materials. The upper surface layer 104 or the intermediate layer 203 is an inelastic material of which a type can be a non-woven fabric, an inelastic non-porous film, an inelastic microporous film, or an inelastic perforated film and is selected as one of PET, PP, PE, PS, Nylon, or a mixture of the above materials. Further, the upper surface layer 104 or the lower surface layer 303 is preferably of a material that comprises PP, polypropylene elastomer, or a mixture of the above materials. Further, the intermediate layer 203 or the lower surface layer 303 comprises a material that is added with inorganic powder of calcium carbonate, magnesium carbonate, oxides of aluminum, or oxides of titanium having a weight percentage of 1-75%. The upper surface layer 104 has a thickness that is 5-70% of an entire thickness; the intermediate layer 203 has a thickness that is 5-70% of the entire thickness; and the lower surface layer 303 has a thickness that is 5-70% of the entire thickness.

A method for manufacturing an elastomeric film composite of the above embodiments comprises: a co-extrusion process, in which a plurality of extrusion machines are operated to feed a plurality of raw materials to extrude, through a multi-layer co-extrusion mold, and form a multi-layer film; and an extension process, in which the co-extruded multi-layer film is subjected to processing through roller speed difference or calendaring to form an interlayer surface gap; and material surface compatibility differences are used to pull open the surface gap between layers through applying the extension process without application of a hot pressing process so that the elastomeric film composite is made to have an embossed surface structure in the form of cloth. Another method for manufacturing the elastomeric film composite is also available, which comprises: at least a lamination process, in which thermoplastic raw materials are molten and extruded through a hot melting process and, at the same time, get adhered to a non-woven fabric base; and an extension process, in which the co-extruded multi-layer film is subjected to processing through roller speed difference or calendaring to form an interlayer surface gap; and material surface compatibility differences are used to pull open the surface gap between layers through applying the extension process without application of a hot pressing process so that the elastomeric film composite is made to have an embossed surface structure in the form of cloth. In these methods, the application of the extension process is carried out by traction through roller speed difference, stretching, pressing roll calendaring, or toothed roller pinching engagement. A direction of extension can be selected to achieve extension in a machine direction (MD), extension in a cross direction (CD), or bi-directional extension. Before the extension process, the elastomeric film composite has a basis weight that is between 30-100 gsm. After the extension process, the elastomeric film composite has a basis weight that is between 30-500 gsm. The extension process is conducted such that extension rate can be destructive or non-destructive to make a film of the elastomeric film composite become a broken condition or a non-broken condition. The extension rate of the extension process is between ≥0 and ≤10. The composite that is in an extended condition after the application of the extension process shows moisture vapor transfer (M.V.T., ASTM E96-BW) between ≥50 g/m²·24 hrs and ≤15000 g/m²·24 hrs. The composite that is in an extended condition after the application of the extension process shows W.R.S., AATCC 127, between ≥12 mmH₂O and ≤2000 mmmH₂O. The composite, after the application of the extension process, has an overall or entire thickness that is between ≥10 micrometers and ≤2000 micrometers. The composite, after the application of the extension process, has a tensile strength (ASTM-D5034, Grab type) between ≥1 Kgf and ≤160 Kgf and elongation (ASTM-D5034, Grab type) between ≥40% and ≤600%.

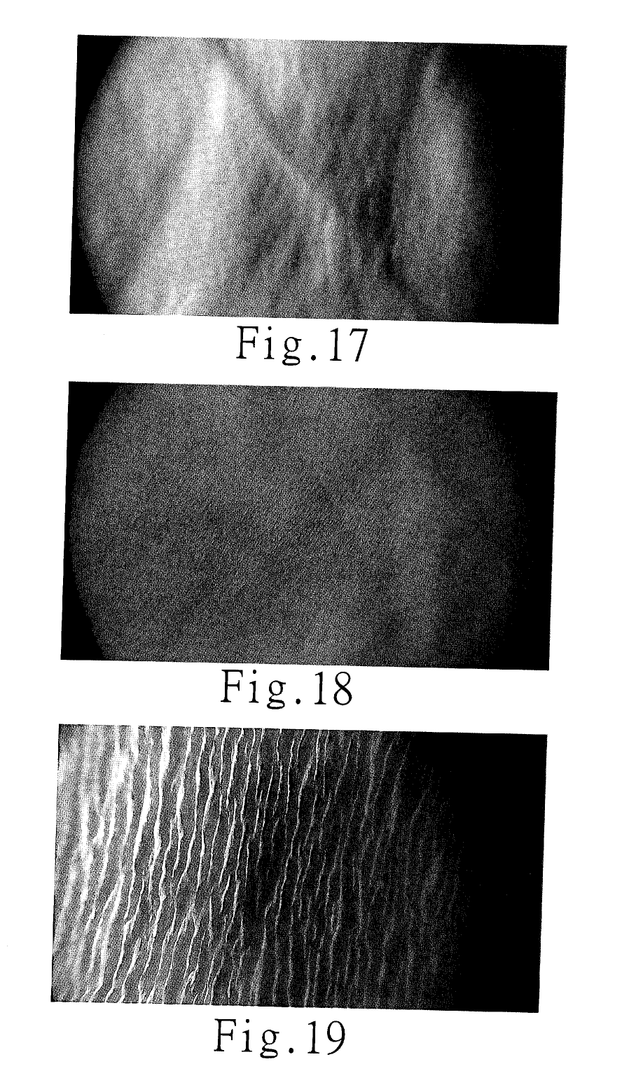

FIG. 17 is a microscopic picture showing a film surface of an elastic intermediate layer microporous film composite having a small-sized surface gap according to the present invention, illustrating a condition in which the surface fluffiness is relatively small, exhibiting a smooth, slightly or tiny bulging state, wherein during the manufacturing process, the extension rate of the extension process can be adjusted to a smaller vale or surface compatibility can be made better such that separation that forms the interlayer surface gap is made hard. FIG. 18 is a microscopic picture showing a film surface of an elastic intermediate layer microporous film composite having a medium-sized surface gap according to the present invention, illustrating a condition in which the surface fluffiness is made moderately large, exhibiting a smooth, moderately tiny bulging state, wherein during the manufacturing process, the extension rate of the extension process can be adjusted to an increased vale or surface compatibility can be made slightly poor such that separation that forms the interlayer surface gap is enhanced. FIG. 19 is a microscopic picture showing a film surface of an elastic intermediate layer macroporous film composite having a large-sized surface gap according to the present invention, illustrating a condition in which the surface fluffiness is relatively large, exhibiting an uneven, raised and recessed, surface state, wherein during the manufacturing process, the extension rate of the extension process can be adjusted to a larger vale or surface compatibility can be made poor such that separation that forms the interlayer surface gap is made easy.

The present invention relates to an elastomeric film composite, which comprises: at least one upper surface layer, at least one intermediate layer, and at least one lower surface layer. Material surface compatibility is selectively made poor and is used to pull open a surface gap between layers through applying an extension process without application of hot pressing so that the elastomeric film composite is made to have an embossed surface structure in the form of cloth. The present invention provides an elastomeric film composite having a unique surface configuration that is different from the prior art and exhibits diversification, as being novel, improved, and utilizable, so as to alleviate the drawbacks and shortcomings of the prior art, making it practically useful.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. An elastomeric film composite, comprising:
at least one upper surface layer, which is adjacent to a first surface of an intermediate layer;
at least one intermediate layer, which comprises the first surface and a second surface and is arranged between the upper surface layer and a lower surface; and
at least one lower surface layer, which is adjacent to the second surface of the intermediate layer;
wherein material surface compatibility difference is used to pull open a surface gap through applying an extension process without application of hot pressing so that the elastomeric film composite is made to have a embossed surface structure in the form of cloth.

2. The elastomeric film composite according to claim 1, wherein the upper surface layer, the intermediate layer, or the lower surface layer is of an elastic material of which a type is an elastic non-porous film, an elastic microporous film, an elastic perforated film, or an elastic non-woven fabric.

3. The elastomeric film composite according to claim 1, wherein the upper surface layer, the intermediate layer, or the lower surface layer comprises an elastic material that is selected as Hytrel, TPU, SEBS, SIS, SBS, polypropylene elastomer, polyethylene elastomer, elastic nylon, or a mixture of the above materials.

4. The elastomeric film composite according to claim 1, wherein the upper surface layer, the intermediate layer, or the lower surface layer comprises an inelastic material of which a type is non-woven fabric, an inelastic non-porous film, an inelastic macroporous film, or an inelastic perforated film.

5. The elastomeric film composite according to claim 1, wherein the upper surface layer, the intermediate layer, or the lower surface layer comprises an inelastic material selected as PET, PP, PE, PS, Nylon, or a mixture of the above materials.

6. The elastomeric film composite according to claim 1, wherein the upper surface layer or the lower surface layer comprises a material that is selected as PP, polypropylene elastomer, or a mixture of the above materials.

7. The elastomeric film composite according to claim 1, wherein the upper surface layer, the intermediate layer, or the lower surface layer comprises a material that is added with inorganic powder having a weight percentage of 1-75%.

8. The elastomeric film composite according to claim 7, wherein the inorganic powder added in the material of the upper surface layer, the intermediate layer, or the lower surface layer is selected as calcium carbonate, magnesium carbonate, oxides of aluminum, or oxides of titanium.

9. The elastomeric film composite according to claim 1, wherein the upper surface layer and the intermediate layer are partly separated from each other.

10. The elastomeric film composite according to claim 1, wherein the lower surface layer and the intermediate layer are partly separated from each other.

11. The elastomeric film composite according to claim 1, wherein the upper surface layer and the intermediate layer are partly separated from each other and the lower surface layer and the intermediate layer are partly separated from each other.

12. The elastomeric film composite according to claim 1, wherein the upper surface layer has a thickness that is 5-70% of an entire thickness.

13. The elastomeric film composite according to claim 1, wherein the intermediate layer has a thickness that is 5-70% of an entire thickness.

14. The elastomeric film composite according to claim 1, wherein the lower surface layer has a thickness that is 5-70% of an entire thickness.

15. The elastomeric film composite according to claim 1, wherein the surface gap has a size of 0.5-1000 micrometers.

16. A method for manufacturing an elastomeric film composite, comprising:
a co-extrusion process, in which a plurality of extrusion machines are operated to feed a plurality of raw materials to extrude, through a multi-layer co-extrusion mold, and form a multi-layer film; and
an extension process, in which the co-extruded multi-layer film is subjected to processing through roller speed difference or calendaring to form an interlayer surface gap;
wherein material surface compatibility difference is used to pull open the surface gap between layers through applying the extension process without application of hot pressing so that the elastomeric film composite is made to have an embossed surface structure in the form of cloth.

17. A method for manufacturing an elastomeric film composite, comprising:
at least a lamination process, in which thermoplastic raw materials are molten and extruded through a hot melting process and, at the same time, get adhered to a non-woven fabric base; and
an extension process, in which the co-extruded multi-layer film is subjected to processing through roller speed difference or calendaring to form an interlayer surface gap;
wherein material surface compatibility difference is used to pull open the surface gap between layers through applying the extension process without application of hot pressing so that the elastomeric film composite is made to have an embossed surface structure in the form of cloth.

18. The method for manufacturing elastomeric film composite according to claim 16, wherein the extension process is carried out by traction through roller speed difference, stretching, pressing roll calendaring, or toothed roller pinching engagement.

19. The method for manufacturing elastomeric film composite according to claim 17, wherein the extension process is carried out by traction through roller speed difference, stretching, pressing roll calendaring, or toothed roller pinching engagement.

20. The method for manufacturing elastomeric film composite according to claim 16, wherein a direction of extension can be selected to achieve extension in a machine direction (MD), extension in a cross direction (CD), or bi-directional extension.

21. The method for manufacturing elastomeric film composite according to claim 17, wherein a direction of extension can be selected to achieve extension in a machine direction (MD), extension in a cross direction (CD), or bi-directional extension.

22. The method for manufacturing elastomeric film composite according to claim 16, wherein before the extension process, the elastomeric film composite has a basis weight that is between 30-100 gsm.

23. The method for manufacturing elastomeric film composite according to claim 17, wherein before the extension process, the elastomeric film composite has a basis weight that is between 30-100 gsm.

24. The method for manufacturing elastomeric film composite according to claim 16, wherein after the extension process, the elastomeric film composite has a basis weight that is between 30-500 gsm.

25. The method for manufacturing elastomeric film composite according to claim 17, wherein after the extension process, the elastomeric film composite has a basis weight that is between 30-500 gsm.

26. The method for manufacturing elastomeric film composite according to claim 16, wherein the extension process is conducted such that extension rate is destructive or non-destructive to make a film of the elastomeric film composite become a broken condition or a non-broken condition.

27. The method for manufacturing elastomeric film composite according to claim 17, wherein the extension process is conducted such that extension rate is destructive or non-destructive to make a film of the elastomeric film composite become a broken condition or a non-broken condition.

28. The method for manufacturing elastomeric film composite according to claim 16, wherein extension rate of the extension process is between ≥0 and ≤10.

29. The method for manufacturing elastomeric film composite according to claim 17, wherein extension rate of the extension process is between ≥0 and ≤10.

30. The method for manufacturing elastomeric film composite according to claim 16, wherein the composite that is in an extended condition after the application of the extension process shows moisture vapor transfer (M.V.T., ASTM E96-BW) between ≥50 g/m²·24 hrs and ≤15000 g/m²·24 hrs.

31. The method for manufacturing elastomeric film composite according to claim 17, wherein the composite that is in an extended condition after the application of the extension process shows moisture vapor transfer (M.V.T., ASTM E96-BW) between ≥50 g/m²·24 hrs and ≤15000 g/m²·24 hrs.

32. The method for manufacturing elastomeric film composite according to claim 16, wherein the composite that is in an extended condition after the application of the extension process shows W.R.S., AATCC 127, between ≥12 mmH₂O and ≤2000 mmmH₂O.

33. The method for manufacturing elastomeric film composite according to claim 17, wherein the composite that is in an extended condition after the application of the extension process shows W.R.S., AATCC 127, between ≥12 mmH₂O and ≤2000 mmmH₂O.

34. The method for manufacturing elastomeric film composite according to claim 16, wherein the composite, after the application of the extension process, has an entire thickness that is between ≥10 micrometers and ≤2000 micrometers.

35. The method for manufacturing elastomeric film composite according to claim 17, wherein the composite, after the application of the extension process, has an entire thickness that is between ≥10 micrometers and ≤2000 micrometers.

36. The method for manufacturing elastomeric film composite according to claim 16, wherein the composite, after the application of the extension process, has a tensile strength (ASTM-D5034, Grab type) between ≥1 Kgf and ≤160 Kgf.

37. The method for manufacturing elastomeric film composite according to claim 17, wherein the composite, after the application of the extension process, has a tensile strength (ASTM-D5034, Grab type) between ≥1 Kgf and ≤160 Kgf.

38. The method for manufacturing elastomeric film composite according to claim 16, wherein the composite, after the application of the extension process, has an elongation (ASTM-D5034, Grab type) between ≥40% and ≤600%.

39. The method for manufacturing elastomeric film composite according to claim 17, wherein the composite, after the application of the extension process, has an elongation (ASTM-D5034, Grab type) between ≥40% and ≤600%.
